(19) United States
(12) Patent Application Publication
(10) Pub. No.: US 2024/0043808 A1
(43) Pub. Date: Feb. 8, 2024

(54) Screenable Liver Disease Models and Methods

(71) Applicant: INSPHERO AG, (US)

(72) Inventors: Simon Messner, Zurich (CH); Jens M Kelm, Zurich (CH); Radina Kostadinova-Angelova, Zurich (CH); Wolfgang Moritz, Bassersdorf (CH)

(21) Appl. No.: 18/231,156

(22) Filed: Aug. 7, 2023

Related U.S. Application Data
(63) Continuation of application No. 16/471,551, filed on Jun. 19, 2019, now abandoned.

(30) Foreign Application Priority Data
Dec. 23, 2016 (GB) 1622149.1

(51) Int. Cl. C12N 5/071; C12N 5/00; G01N 33/50

(52) U.S. Cl. CPC C12N 5/0671; C12N 5/0062; C12N 5/0697; G01N 33/5067; G01N 33/5088; C12N 2503/04; C12N 2513/00; G01N 2800/52

(57) ABSTRACT

An in vitro testing platform for the study of metabolic liver disease and related therapeutic strategies, composed of an artificial spheroidal microtissue comprising at least hepatocytes and hepatic stellate cells, and at least one type of hepatic inflammatory cells and further medium and reagents capable to establish and simulate different stages of metabolic liver disease and their progression and testing preventive and therapeutic strategies by pharmacological and/or dietary interventions.

### RELATED APPLICATIONS

This application is a continuation of U.S. patent application Ser. No. 16/471,551 filed Jun. 19, 2019, which claims the benefit of priority under 35 U.S.C. 371 of International Patent No. PCT/EP2017/058174 filed Dec. 27, 2017, which claims the benefit of priority from United Kingdom Patent Application No. 1622149.1 filed Dec. 23, 2016.

### FIELD OF THE INVENTION

The present invention relates to the field of 3D tissue cultures and its applications in drug testing.

### INTRODUCTION

Liver fibrosis is a common outcome of virtually all chronic hepatic insults including viral hepatitis (i.e., hepatitis B and C), alcoholic or obesity-associated steatohepatitis (i.e., nonalcoholic steatohepatitis (NASH)), parasitic disease (i.e., schistosomiasis), metabolic disorders (i.e., Wilson's), hemochromatosis and other storage diseases, congenital abnormalities, autoimmune and chronic inflammatory conditions (i.e., sarcoidosis), and drug toxicity, among others (Puche, Saiman, & Friedman, 2013).

Liver Fibrosis is defined by the World Health Organisation as the presence of excess collagen due to new fiber formation. The fibers result from production of extracellular matrix (ECM) due to activation of quiescent hepatic stellate cells into myofibroblasts.

Liver fibrosis is currently mainly studied in animal experiments, but also isolated cell types of the liver are employed to study fibrosis in-vitro. In-vivo experimental models of liver fibrosis summarized in the review from Liedtke et al., 2013. Briefly, the most common animal model for liver fibrosis is the surgical bile-duct ligation. Other models employ genetic engineered animals, such as Mdr2 knockout mice or dominant negative TGF-βRII mice. Another type of animal models induces liver fibrosis through injection of chemicals (e.g. Carbon Tetrachloride, Thiocetamide, Dimethylnitrosamine) or through feeding (e.g. high-fat diet, choline deficient diet).

Currently employed in-vitro models for liver fibrosis typically use 2D-cultures of hepatic stellate cell lines (HSC, e.g., hTERT-HSC, LX2) or freshly isolated human or rodent HSC. The common limitation of these culture systems are that the HSC activate in culture into myofibroblasts, due to the interaction of HSC with the 2D culture dish. In addition, these models are just an incomplete representation of the liver's morphology and physiology, which limits their use in preclinical applications, e.g., assaying agents for their risk to cause fibrosis.

In recent years, the use of 3D spheroid cultures has become a more applicable format to obtain biologically relevant in vitro liver system for the study of liver disease (van Grunsven, 2017). However, current state of the art does not describe the reconstitution of liver organoids through the assembly of the four major liver cell types, i.e. hepatocytes, Kupffer cells, stellate cells and endothelial cells, and its use for the study of human liver disease, in particular but not exclusively the progression from healthy liver to NASH/fibrosis.

### SUMMARY OF THE INVENTION

It is one object of the present invention to provide a liver disease model which has a superior physiological behavior.

It is one further object of the present invention to provide a liver disease model which is suitable for studying any of Steatosis, Inflammation, Fibrosis and Cirrhosis and respective therapeutic strategies.

It is one further object of the present invention to provide a liver disease model which is suitable for high throughput screening purposes.

These and further objects are achieved by the subject matter of the independent claims, while the dependent claims as well as the specification disclose further preferred embodiments.

### EMBODIMENTS OF THE INVENTION

Before the invention is described in detail, it is to be understood that this invention is not limited to the particular component parts or structural features of the devices or compositions described or process steps of the methods described as such devices and methods may vary. It is also to be understood that the terminology used herein is for purposes of describing particular embodiments only, and is not intended to be limiting. The mere fact that certain measures are recited in mutually different dependent claims does not indicate that a combination of these measures cannot be used to advantage. Any reference signs in the claims should not be construed as limiting the scope. It must be noted that, as used in the specification and the appended claims, the singular forms “a,” “an” and “the” include singular and/or plural referents unless the context clearly dictates otherwise. Further, in the claims, the word “comprising” does not exclude other elements or steps.

It is moreover to be understood that, in case parameter ranges are given which are delimited by numeric values, the ranges are deemed to include these limitation values.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature also for the other embodiment, but that just for purposes of clarity and to keep the specification in a manageable volume this has not been done.

Furthermore, the content of the prior art documents referred to herein is incorporated by reference. This refers, particularly, for prior art documents that disclose standard or routine methods. In that case, the incorporation by reference has mainly the purpose to provide sufficient enabling disclosure, and avoid lengthy repetitions.

According to one embodiment of the microtissue (MT) of the invention, an artificial spheroidal microtissue comprising at least hepatocytes and hepatic stellate cells, and further at least one type of hepatic inflammatory cells, is provided.

As used herein, the term “hepatic inflammatory cells, refers to cells in the liver that mediate inflammation, or are activated by inflammatory cytokines. Such cells are, for example, invariant natural killer cells, T lymphocytes, polymorphonuclear cells, or CD8 lymphocytes or a mixture thereof such as Peripheral Blood Mononuclear Cells (PBMCs).

Microtissues mimicking hepatic organoids have been disclosed in Leite et al., 2016. The organoids described therein consist of (i) hepatocytes derived from HepaRG hepatic stem cells plus (ii) hepatic stellate cells (HSC). The authors describe the use of organoids comprising these two cell types in testing drug-induced liver fibrosis in vitro.

The authors claim that their hepatic organoid culture model is the first that can detect hepatocyte-dependent and compound induced HSC activation, thereby representing an important step forward towards in vitro compound testing for drug-induced liver fibrosis.

However, Leite et al. do not disclose the use of inflammatory cells in the organoids they describe.

According to one embodiment of the microtissue according to the invention, the hepatic inflammatory cells are Kupffer cells. The inventors of the present invention have realized that Kupffer cells (also known as Kupffer-Browicz cells) play an important role in the transition of fatty liver towards steatohepatitis and eventually into liver fibrosis (Duarte et al., 2015). They have realized that the use thereof in a spheroidal microtissue can contribute to generate better organoids for use in liver fibrosis research.

Kupffer cells are specialized macrophages located in the liver, where they form part of the mononuclear phagocyte system. Kupffer cell activation is not only responsible for early ethanol-induced liver injury, common in chronic alcoholics, but also in non-alcoholic fatty liver disease (NAFLD) promoting an inflammatory response which contributes to the development of non-alcoholic steatohepatitis (NASH), which has been recognized as major cause for liver fibrosis. Kupffer cells hence play an important role in the progression of NAFLD towards NASH and liver fibrosis.

In order to further increase the quality of the artificial spheroidal microtissue with respect to its usability as a liver NASH/fibrosis model, the inventors therefore suggest, for the first time, to incorporate also Kupffer cells into such model.

One other type of liver microtissue is provided by Organovo, and called “exVive 3D Liver”. These tissue models are created using 3D bioprinting technology and different liver cell types. The resulting tissues contain a reproducible architecture and are being used for liver tissue-specific toxicity marker assessment as a supplement to in vitro and preclinical (non-GLP) animal testing.

For bioprinting these microtissues, bioinks are being prepared which comprise the cells that later form the tissues. The bioinks are then deposited in a two-compartment planar geometry onto the membranes of standard 24-well plates via continuous deposition microextrusion with non-parenchymal cells comprising the border regions of each compartment and the parenchymal cells filling each compartment (see Nguyen et al, 2016).

The tissues obtained by said 3D bioprinting process do not have a spheroidal shape—they rather come in cylindric shapes, and do hence have disadvantages as regards their physiological behavior.

The spheroidal shape cannot be produced with 3D bioprinting processes, but largely relies on self assembly of the cells, as will be described below. The spheroidal shape makes sure that cells receive enough oxygen and nutrients since the tissue diameter is below 500 μm. The small size of the spheroids needs only small amounts of the precious primary cell material and thus allows production of many spheroids from a limited amount of cellular material.

According to one embodiment of the microtissue of the invention, the microtissue further comprises endothelial cells.

The endothelial cells act as a selective permeability barrier, in such way that they control which molecules enter, from the blood vessels, into the surrounding tissue including the hepatocytes.

Endothelial cells facilitate access of the hepatocyte to oxygen and small molecules in the microcirculation. Other specialized functions include clearance of colloids and macromolecules, promotion of hepatic stellate cell quiescence, and induction of immune tolerance.

The hepatic endothelial cell may be injured by a variety of toxins, ischemia-reperfusion, and even bacteria, leading to vascular liver diseases such as fibrosis.

Preferably, the endothelial cells are sinusoidal endothelial cells. Liver sinusoids are a type of sinusoidal blood vessel with fenestrated, discontinuous endothelium that serve as a location for mixing of the oxygen-rich blood from the hepatic artery and the nutrient-rich blood from the portal vein. Hepatocytes are separated from the sinusoids by the space of Disse.

Furthermore, the inventors have realized that sinusoidal endothelial cell keep quiescent in vivo, hence avoiding that they activate into myofibroblast. Hence, sinusoidal endothelial cell increase cellular life span within the microtissues and thus treatment time.

Sinusoidal endothelial cells play thus an important role in liver physiology and, as such, in liver fibrosis pathogenesis. In order to increase the quality of the artificial spheroidal microtissue with respect to its usability as a liver fibrosis model, the inventors therefore suggest, for the first time, to incorporate also sinusoidal endothelial cells into such model.

The incorporation of sinusoidal endothelial cells into spheroidal microtissues that comprise hepatocytes and stellate cells provides many technical challenges.

Further, it is a challenge to ensure that the liver sinusoidal endothelial cells (LEC) grow on the outer face of the spheroidal microtissues. In a mixed culture. LEC grow interspersed with the remaining liver cells, which would not sufficiently mimic the microanatomy of liver tissue. The inventors have hence developed a suitable coating protocol which makes sure that the LEC grow on the outside of the spheroidal microtissues, and hence can develop their function as endothelial cells.

According to one further embodiment of the microtissue of the invention, the microtissue has a diameter of between ≥30 μm and ≤500 μm. Preferably, the diameter is ≤450 μm, more preferably ≤400 μm, even more preferably ≤350 μm and most preferably ≤300 μm.

According to one further embodiment of the microtissue of the invention, the microtissue comprises between ≥500 and ≤10000 cells in total. Preferably, the microtissue comprises ≤8000 cells, more preferably ≤6000 cells, even more preferably ≤5000 cells and most preferably ≤3000 cells.

The exVive 3D Liver tissues discussed above have, next to their cylindrical shape, diameters of about 2.5 mm, and a thickness of about 0.4 mm (Organovo white paper of 2015), which makes them unsuitable for use in High Throughput Screening (HTS) environments, which usually require microtiter plates with 384 wells or more (see the plate types marked with an asterisk in the following table).

| Type of | Well diameter |
|---|---|
| microtiter plate | (bottom, mm) |
| 24 | 16 |
| 96 | 6.4 |
| 384* | 3.0 |
| 1536* | 1.6 |
| 3456* | <1 |

Both the shape and the minimum size of these tissues are defined by the bioprinting technique.

In contrast thereto, the spheroidal microtissues according to the invention can be made with diameters of between ≥30 μm and ≤500 μm. Besides other advantages, including better oxygen transfer and better transparency for microscopical or video imaging applications, this size range allows their use in microtiter plates with 384, 1536 or even 3456 wells, making them suitable for HTS applications as well.

Furthermore, the exVive 3D Liver tissues comprise up to 10⁶ cells in total, which corresponds to approx. 300-400 times the cell number required for one liver microtissue of a size of 250 μm in diameter. Current bioprinting formats are much more demanding regarding to cell sourcing and associated costs. In this context it is important to understand that the provision of sufficient cell mass is a critical issue in primary cell culture, in particular when spheroidal 3D microtissues are made. This scrutiny is even fostered by the fact that in many cases, the cells used for spheroidal 3D microtissues are primary human cells that are derived from organ donations.

Generally, organs are used which do not qualify for therapeutic transplantation. However even these organs are in extremely short supply.

The spheroidal microtissues according to the invention have lower demands as regards resources. In fact, roughly two order of magnitude less cells are being required for microtissue formation. This means that more microtissues can be made from less donated livers.

According to one embodiment of the microtissue of the invention, the sinusoidal endothelial cells form a cell layer around the three dimensional microtissue comprising at least the hepatocytes and the hepatic stellate cells.

In this embodiment, the sinusoidal endothelial cells can efficiently act as a selective permeability barrier, in such way that they control which molecules enter, from the medium, into the artificial spheroidal microtissue including the hepatocytes, the stellate cells and, optionally, the Kupffer cells. Preferably, the sinusoidal endothelial cells form a monolayer on the microtissue surface.

According to one embodiment of the microtissue of the invention, the cells in the microtissue are mammalian cells, preferably selected from the group consisting of
human cells
monkey cells
pig cells
canine cells, and/or
rodent cells.

According to one other embodiment of the microtissue of the invention,
a) the microtissue comprises pro-activated stellate cells, and/or
b) the microtissue represents a liver model at basal state.

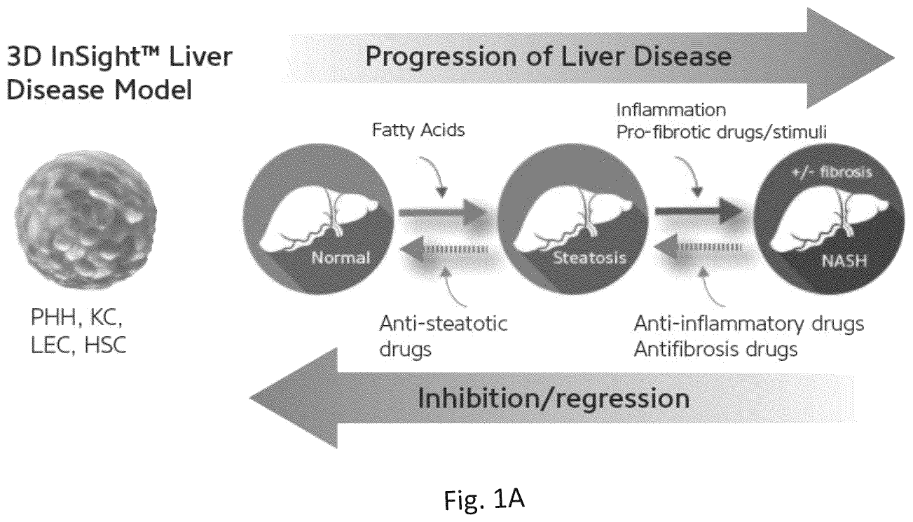

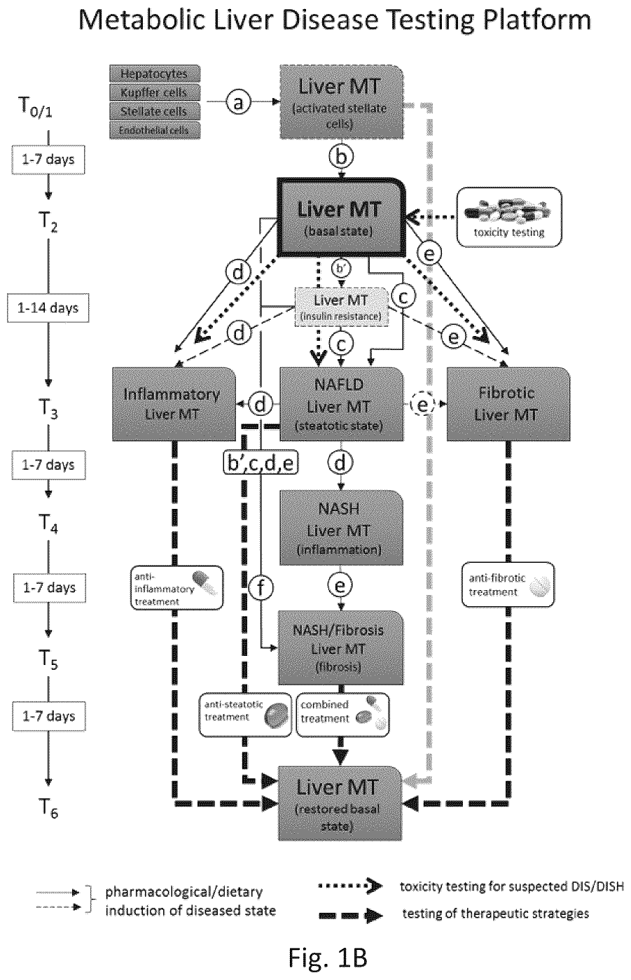

As regards option a) such microtissue can be obtained, e.g., at time T1, i.e., right after tissue formation. See process [a] as shown in FIG. 1B.

As regards option b) such microtissue can be obtained, e.g., after reversion from an activated post-production state. See process [b] as shown in FIG. 1B, and as e.g. described in example 8.

According to one further embodiment of the microtissue of the invention, the microtissue is progressed to at one of the states selected from the list consisting of:
a) insulin resistant state,
b) steatotic state,
c) inflammatory state,
d) fibrotic state,
e) NASH/Fibrosis phenotype, option 1,
f) NASH/Fibrosis phenotype, option 2.

As regards option a), state can be induced, e.g., by, process [b′], e.g., by exposure to
high glucose/fructose
high insulin
high free fatty acids (SFA>MUFA>PUFA)
adipocyte derived cytokines TNF-α, IL-1β, and IL-6
resistin
glucocorticoids
or a combination of above

As regards option b), such state can be induced, e.g., by process [c] as shown in FIG. 1B, and as e.g. described in example 3, with. e.g., a MUFA:SFA molar ratio ≥1

As regards option c), such state can be induced, e.g., by process [d] as shown in FIG. 1B, and as e.g. described in example 4.

As regards option d), such state can be induced, e.g., by process [e] as shown in FIG. 1B, and as e.g. described in example 4 and 5.

As regards option e), such state can be induced, e.g., by sequential or simultaneous application of process [b′], [c], [d], and [e] as shown in FIG. 1B.

As regards option f), such state can be induced, e.g., by treatment with pro-inflammatory dietary free fatty acid composition (see, e.g., process [f] as shown in FIG. 1B.), with e.g. SFA only or MUFA:SFA molar ratio <1.

The term “PUFA”; as used herein, means polyunsaturated fatty acids or fats. The term “MUFA”; as used herein, means monounsaturated fatty acids or fats. The term “SFA”; as used herein, means saturated fatty acids or fats.

Most common SFAs in human diet are: Stearic acid, Palmitic acid, Myristic acid, Lauric acid, Capric acid, Caprylic acid, Caproic acid. Most common MUFAs in human diet are: Oleic acid, Palmitoleic acid.

According to one further aspect of the invention, a method of preparing a spheroidal microtissue comprising at least hepatocytes, hepatic stellate cells, hepatic inflammatory cells, and endothelial cells is provided, which method comprises the steps of:
a) producing or providing a microtissue comprising at least hepatocytes, hepatic stellate cells and hepatic inflammatory cells,
b) producing or providing a suspension comprising sinusoidal endothelial cells,
c) dispensing said suspension into wells or vessels comprising one or more microtissues of step a), and
d) incubating said wells or vessels to let sinusoidal endothelial cells adhere to the microtissues.

In said process, the endothelial cells sink down into the wells or vessels comprising the microtissues after dispension or pipetting, and get in contact with the microtissues, where they adhere to the extracellular matrix and from cell-to-cell contacts.

In one embodiment, the endothelial cells are sinusoidal endothelial cells.

According to one embodiment of the method of the invention, the microtissues are grown and cultivated in a hanging drop system.

Such hanging drop system is for example provided by InSphero's GRavityPLUS™ Hanging Drop System (CH), described in WO2010031194A, the content of which is incorporated by reference herein. Methods of making microtissues in such hanging drop system are e.g. disclosed in WO2015158777A1, the content of which is incorporated by reference herein.

According to one further embodiment of the method of the invention, the microtissues are grown and cultivated in a low adherence well.

Such low adherence well system is for example InSphero's GravityTRAP™ ULA Plate (CH). As an alternative, Microtiter plates treated with a cell repellent as for example provided by Greiner can be used. Methods of making microtissues in such system are e.g. disclosed in PCT/EP2016/065571, the content of which is incorporated by reference herein.

According to one further embodiment of the method of the invention, at least some of the cells are cryopreserved cells which are thawed before processing, i.e., before producing the microtissues. According to one other embodiment of the method of the invention, at least some of the cells are non-frozen cells.

According to one further aspect of the invention, a spheroidal microtissue is provided which has been obtained with a method according to the above description.

Such microtissue comprises at least hepatocytes, hepatic stellate cells and hepatic inflammatory cells (plus optionally an outer layer comprising sinusoidal endothelial cells).

It may have a diameter of between ≥30 μm and ≤500 μm. Preferably, the diameter is ≤450 μm, more preferably ≤400 μm, even more preferably ≤350 μm and most preferably ≤300 μm.

It may comprise between ≥500 and ≤10000 cells in total. Preferably, it comprises ≤8000 cells, more preferably ≤6000 cells, even more preferably ≤5000 cells and most preferably ≤3000 cells.

According to one further aspect of the invention, a model platform for liver disease modelling is provided, said model platform comprising a microtissue according to any one of claims 1-9 and 15

Such platform is capable to allow following investigative modes:
1. Study of disease progression and testing of anti-NASH/Fibrosis therapeutic or preventive strategies by pharmacological and/or dietary interventions, starting from different entry points.
2. Safety assessment of drug candidates with suspected liver toxic liabilities

In one embodiment, the model platform allows at least one selected from the group consisting of:
mechanistic studies of disease progression,
prevention of disease progression by pharmacological or dietary intervention, and/or
the reversion of diseased states.

In another embodiment, the model platform is provided in an assay-ready format composed of basic media and reagents enabling the installment and recapitulation of disease specific states, representative of specific preventive and therapeutic entry points.

In one embodiment, the platform further comprises quality-control data and calibration data, which data are specific to the production batch, and provide information about at least one feature selected from the group consisting of:
Biomarkers for disease progression,
Cell viability and functionality, and/or
Microtissue size.

In still another embodiment of the model platform, the liver disease is at least one selected from the group consisting of:
liver fibrosis,
viral hepatitis (e.g., hepatitis B and C),
alcoholic or obesity-associated steatohepatitis (e.g., nonalcoholic steatohepatitis (NASH)),
parasitic disease (e.g., schistosomiasis),
metabolic disorders (e.g., Wilson's),
hemochromatosis and other storage diseases,
congenital abnormality,
autoimmune and chronic inflammatory conditions (e.g., sarcoidosis), and/or
drug toxicity.

In yet another embodiment of the model platform, the spheroidal microtissue is in co-culture with at least one type of inflammatory cells.

In specific embodiments, such inflammatory cells are selected from the group consisting of:
NK, NKT or iNKT cells,
T lymphocytes,
polymorphonuclear cells,
CD8 lymphocytes, and/or
Peripheral Blood Mononuclear Cells (PBMCs).

In one embodiment, at least one type of inflammatory cells has been haplotype-matched to liver cells of the microtissue.

In one further embodiment the microtissue and at least one type of inflammatory cells are
in a static co-culture, or
in a microfluidic device allowing free floating of inflammatory cells yet immobilized liver microtissues

The versatility of this platform allows different modes of implementation or commercialization:
1. Provision of aforementioned basal liver models and disease models as assay ready system, including
   Basal maintenance medium
   Diseased state-specific media
   QC certification
   Reference test compounds
2. Provision of aforementioned basal liver models and supplements for the induction of any of above mentioned disease states as assay-ready system, including
   Basal maintenance medium
   QC certification
   Reference test compounds

According to one further aspect of the invention, a liver model is provided which has been obtained with a method according to any to the above description and/or comprises a spheroidal microtissue according to the above description and/or comprises a platform according to the above description.

According to one further aspect of the invention, an assay method for testing compounds
suspected for promoting, causing, or increasing the risk of NAFLD/NASH/Fibrosis, or
for their potency to reduce the risk, alleviate the symptoms, treat the causes of NAFLD/NASH/Fibrosis or to reverse the pathological states of NAFLD/NASH/Fibrosis,
   is provided, in which assay a spheroidal microtissue according to the above description, or being been obtained with a method according to the above description, and/or comprising a platform according the above description is provided, which assay method comprises the steps of
a) treatment of one or more test microtissues with one or more test compounds suspected for promoting, causing, or increasing the risk of, NAFLD/NASH/Fibrosis, and
b) parallel or subsequent treatment with one or more test compounds intended to reverse NAFLD/NASH/Fibrosis
c) carrying out an endpoint analysis of at least one genetic, physiologic and/or morphologic parameter relevant to the respective liver pathology.

In specific embodiments, said liver fibrosis is NAFLD or NASH/fibrosis. In specific embodiments, the said compounds suspected for promoting, causing, or increasing the risk of liver fibrosis are suspected to induce steatosis (DIS: drug-induced steatosis) or steatohepatitis (DISH: drug-induced steatohepatitis)

According to one further embodiment of said method of the invention, a positive control is furthermore used, which positive control comprises:
a′) treatment of a control microtissue with a control agent selected from a group of fibrosis inducers (e.g. TGF-ß1, methotrexate (MTX) or thioacetamide (TAA), steatosis inducers (e.g. cholesterol, fatty acids, amiodarone, tetracyclin, valproic acid) and/or inflammation inducers (e.g. TNF-α, Lipopolysaccharide, MTX)
b′) carrying out the same endpoint analysis of at least one genetic, physiologic and/morphologic parameter, and

According to one further embodiment of said method of the invention the endpoint analysis refers to at least one parameter selected from the group consisting of:
Release of one or more biomarkers, as for example measured by specific ELISAs, biochemical assays or by omics technologies, e.g. metabolomics, lipidomics, glycomics, and secretomics
Lipid content of the microtissue cells
Histology of the microtissue and its cells
RNA expression marker such as alpha-smooth muscle actin, COL1A1, Vimentin or TIMP-1
Size or volume of the microtissue, and/or
Mitochondrial activity

Example classes of biomarkers for fibrosis include, inter alia, collagen release, fibronectin release, matrix metalloproteinase (MMP) release, tissue inhibitor of matrix metalloproteinase (TIMP) release, cytokine release.

Lipid content of the microtissue cells can be determined by Nile-red or Bodipy (boron-dipyrromethene) staining of triglycerides in microtissues and quantification, or Triglyceride quantification based on biochemical assays.

Histology of the microtissue and its cells can be determined by determination or measurement of ECM-proteins (Collagen 1A1, Collagen 3A1, Collagen 4A1, Laminin, Elastin, Fibronectin, CD44), Stellate cell markers (alpha-SMA, PDGFR-β, Vimentin, Desmin, GFAP), Kupffer cell markers (CD68, CD163), Hepatocyte markers (Albumin, CYP3A4), LEC-markers (CD31) or Steatosis markers (H&E staining; appearance of unstained, white droplets of small to large size in cytoplasma of hepatocytes or Oilred O stained cryosections).

According to one other aspect of the invention, the use of a spheroidal microtissue according to the above description, or obtained with a method according to the above description, and/or comprising a platform according to the above description, for at least one purpose selected from the group consisting of:
drug efficacy and/or toxicity screenings
investigative/mechanistic toxicology,
target discovery/identification,
drug repositioning studies, and/or
pharmacokinetics and pharmacodynamics assays.
   is provided.

### Experiments and Figures

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### FIGURES

FIG. 1A: Mechanism of development of fibrosing non-alcoholic steatohepatitis (NASH). Key stimuli: accumulation of lipids (steatosis) and inflammation (triggered by activation of Kupffer cells). Key cell types: primary human hepatocytes (PHH), Kupffer cells (KC), Liver endothelial cells (LEC) and hepatic stellate cells (HSC). NASH is the intricate interaction of hepatocytes, Kupffer cells and stellate cells and long-term maintenance of signaling pathways leading to fibrosis.

FIG. 1B: Platform according to the invention, in which the different liver states can be induced as described. The following sub-processes are provided:
Liver model with pro-activated stellate cells (T₁, right after tissue formation, process [a])
Liver model at basal state, after reversion from activated post-production state (process [b], described in example 8), or further progressed to either of the following states:
   Liver model with insulin resistant state (process [b′]) introduced by
      High glucose/fructose
      High insulin
      High free fatty acids (SFA>MUFA>PUFA)
      Adipocyte derived cytokines TNF-α, IL-1β, and IL-6
      Resistin
      Glucocorticoids
      or a combination of above
   Liver model with steatotic state, (process [c], described in example 3).
      MUFA:SFA molar ratio ≥1
      Liver model with inflammatory state, (process [d], described in example 4).
      Liver model with fibrotic state, (process [e], described in example 4 and 5).
      Liver model with NASH/Fibrosis phenotype, (by sequential or simultaneous application of process [b′], [c], [d], and [e]).
      Liver model with NASH/Fibrosis phenotype, by treatment with pro-inflammatory dietary free fatty acid composition (process [f])
         SFA only
         or MUFA:SFA molar ratio <1

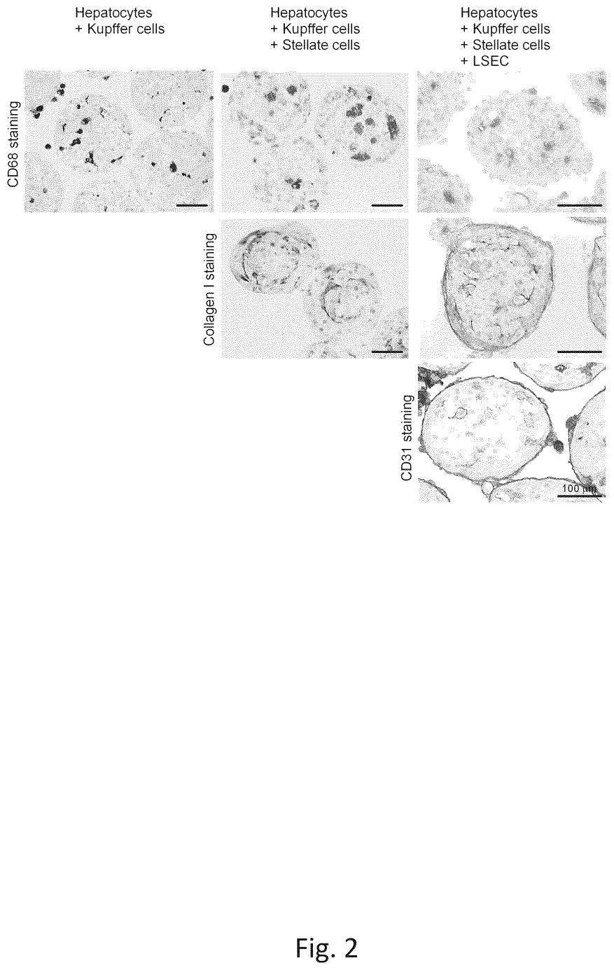

FIG. 2: Presence of the Hepatocytes, Kupffer cells, stellate cells and LEC's in human liver spheroids. FIG. 2 shows photomicrographs of microtissues made with the method according to the present invention, in a hanging drop system. CD68 is a glycoprotein which binds to low density lipoprotein, and is expressed, inter alia, on Kupffer cells. Collagen I is a marker for hepatic stellate cells, while CD31 is a protein that in humans is encoded by the PECAM1 gene, and makes up a large portion of endothelial cell intercellular junctions, hence a marker for endothelial cells. These cells have been coated to the microtissues in a two-step production process to form an endothelial layer between liver tissue and medium.

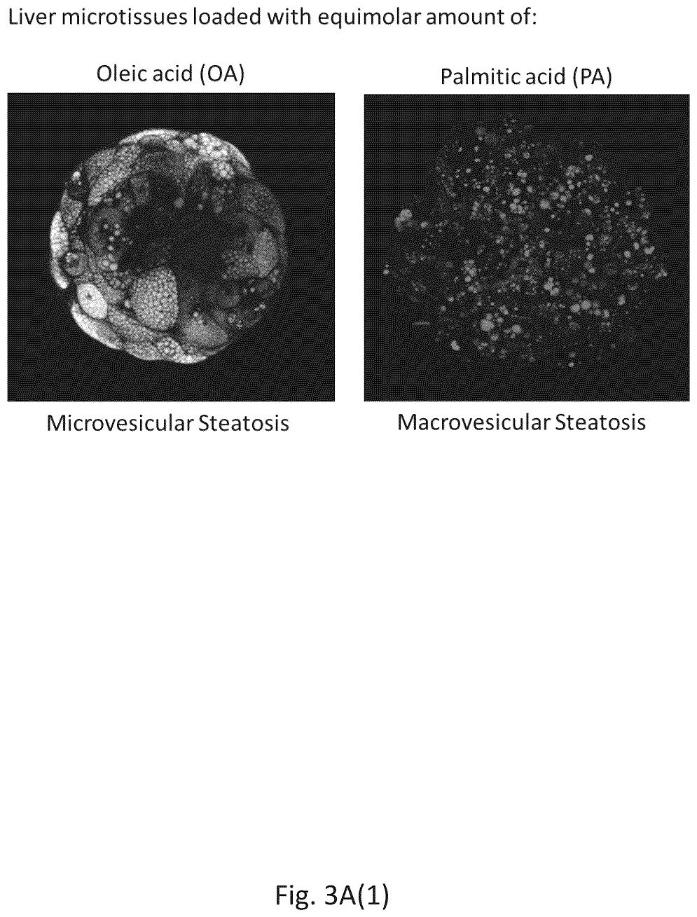

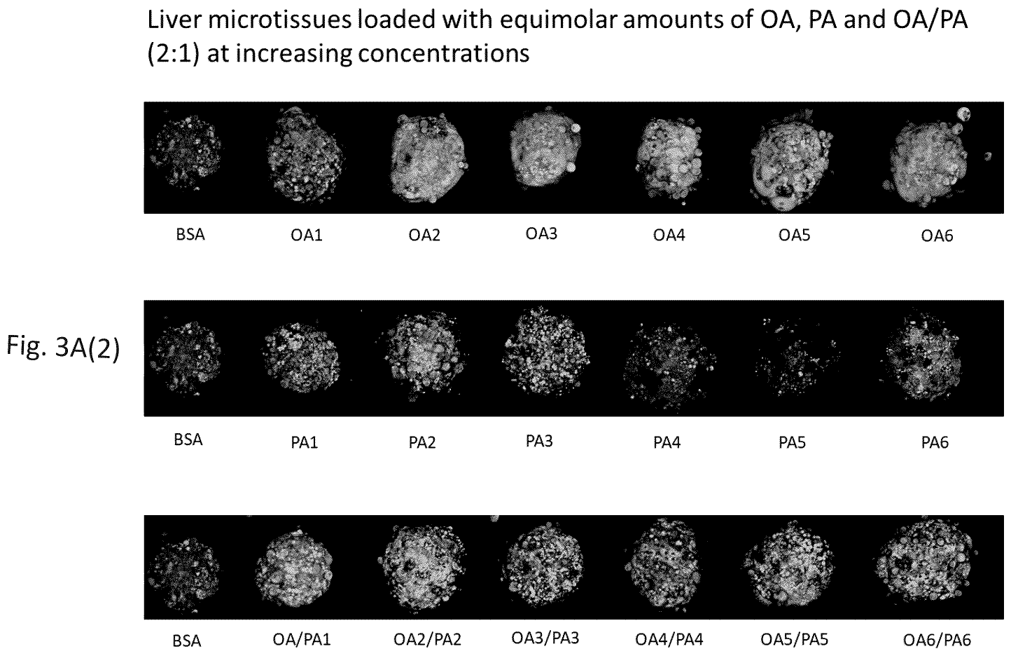

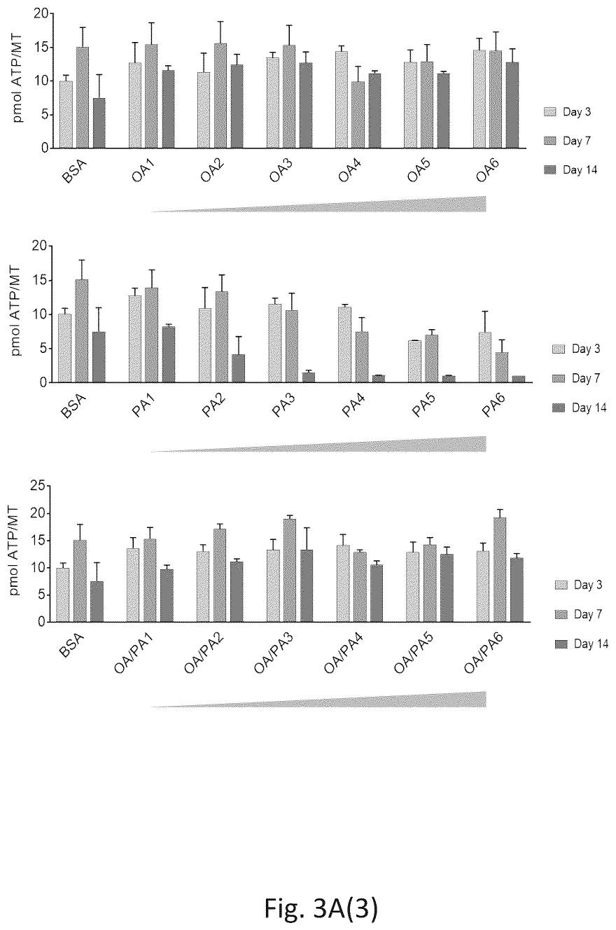

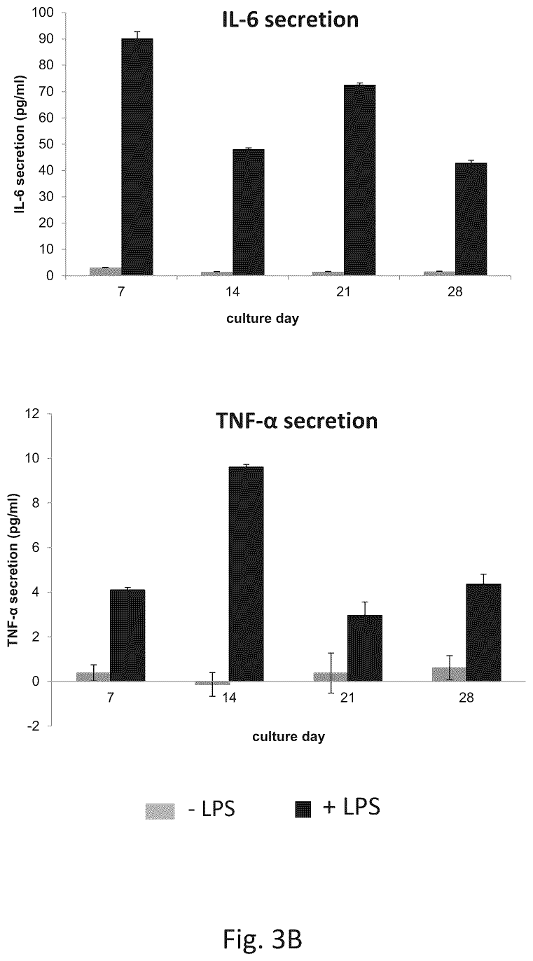

FIG. 3A(1): Induction of liver-disease phenotype “steatosis” as determined by lipid accumulation. Exposure to free fatty acids (Oleate and Palmitate) induced lipid accumulation in microtissues, visualized by nile-red staining and subsequent confocal fluorescence imaging. Images are depicted as maximal intensity projections.

FIG. 3A (2): Lipid accumulation in dependency of increasing concentrations of PA, OA or a combination thereof in a molar ratio of 2:1. The total amount of FFA was identical among the groups at a given concentration.

FIG. 3A(3): ATP measurements from identical groups after exposure to increasing concentrations of FFA for 3-14 days.

FIG. 3B: Induction of liver-disease as determined by secretion of inflammatory cytokines. Human Liver Microtissue co-cultures were treated with lipopolysaccharides for 48 hours once per week for a total of 4 weeks (28 days). The supernatants were analysed for IL-6 and TNF-α induction. Inflammation is thought to be the second step towards recapitulating NASH in vitro

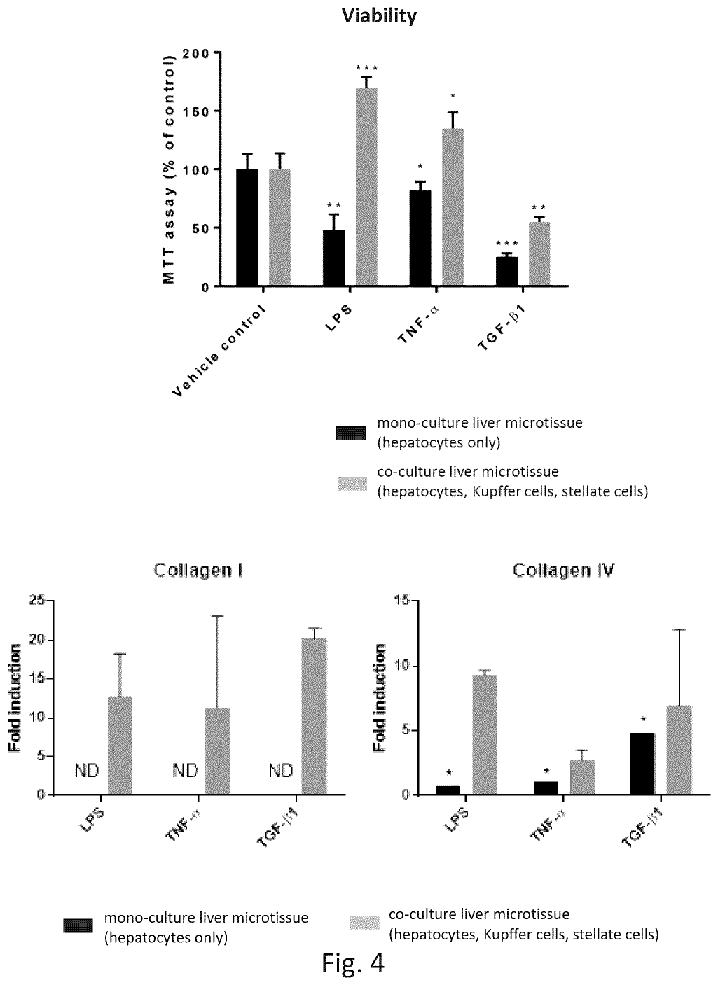

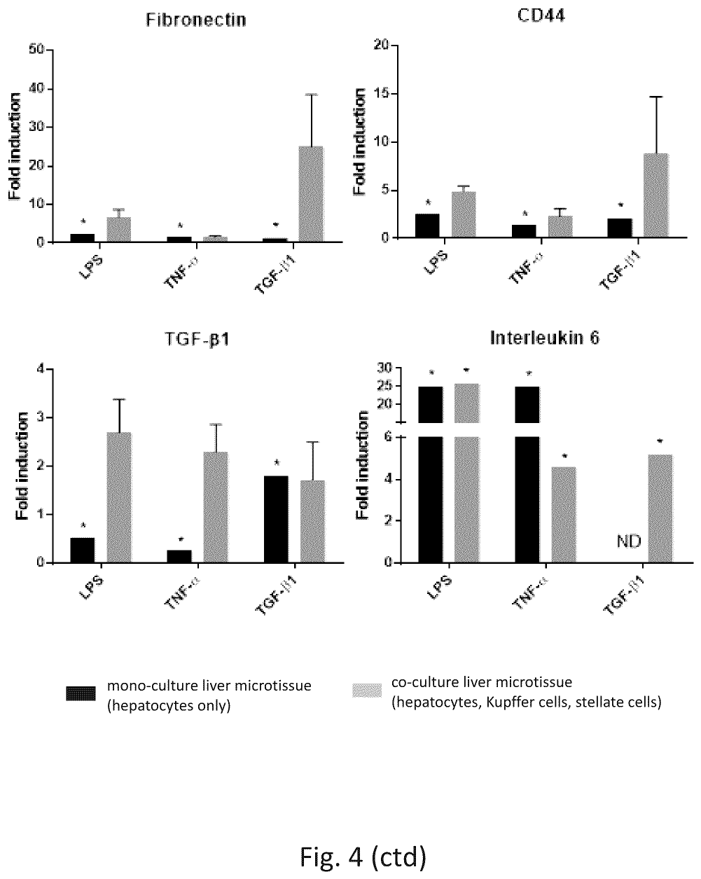

FIG. 4: MTT assay shows enhanced viability in liver cell triple-cultures as compared to hepatocyte mono-culture in presence of LPS and pro-inflammatory cytokines, TNF-α and TGF-β1 (Black bars: hepatocyte mono-culture. Grey bars: hepatocyte+inflammatory cells+stellate cells co-culture). Induction of fibrosis biomarkers, Collagen I and IV upon treatment with inflammation-stimuli LPS or TNF-α and pro-fibrotic TGF-β1 signaling molecule. Gene expression of fibrotic markers (Fibronectin, CD44) and cytokines (TGF-β1, IL-6) in human liver microtissues exposed to LPS, TNF-α and TGF-β1. mRNA was extracted using TRIzol conventional procedure and fold induction were calculated as 2\hat{ }(−ΔΔCT) for each sample and vehicle control and expressed as mean fold induction±S.E.M of three replicates with six MTs each. Actin was used as reference gene for each sample. ND: no-detected values. *; P≤0.05.

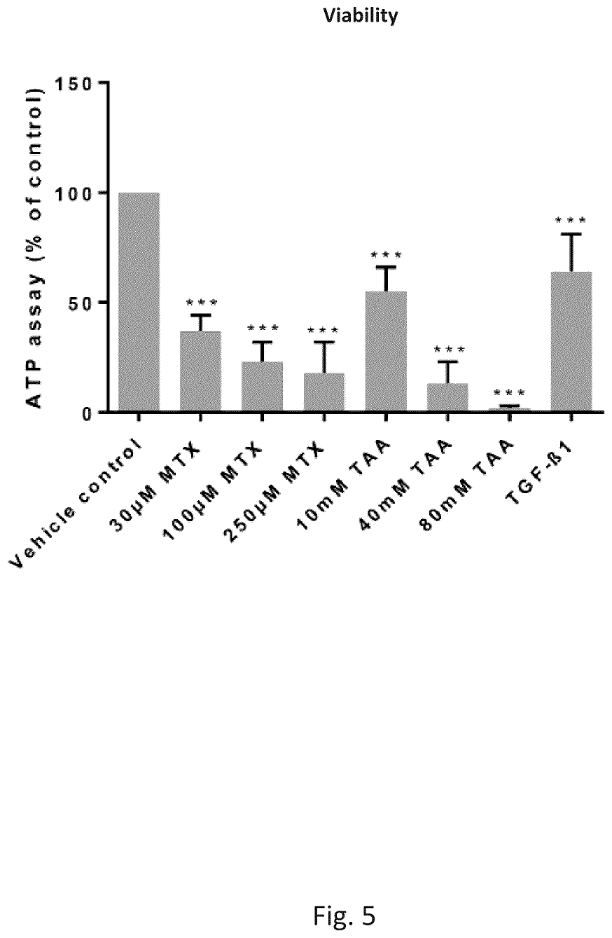

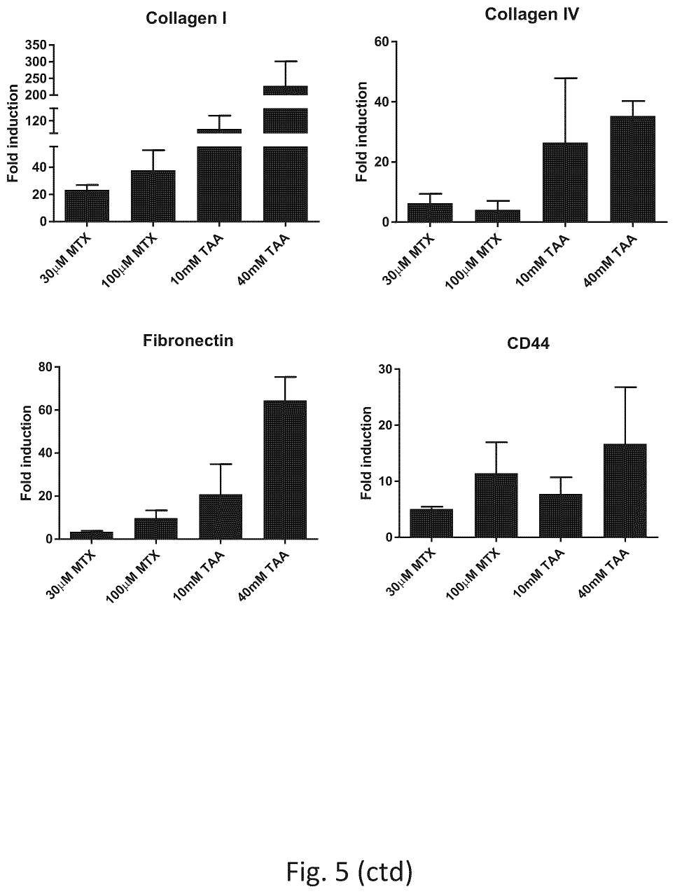

FIG. 5: Dose-dependent loss of viability or metabolic activity observed in liver MT triple-culture (hepatocyte+inflammatory cells+stellate cells) upon treatment with pro-fibrotic compounds MTX and TAA for 14 days, while pro-fibrotic gene expression (Collagen I, Collagen IV, CD44, Fibronectin) was greatly enhanced. MT mRNA was extracted using TRIzol conventional procedure and fold induction were calculated as 2\hat{ }(−ΔΔCT) for each sample and negative control and expressed as mean fold induction±S.E.M. of three replicates with six MTs each. Actin was used as reference gene for each sample.

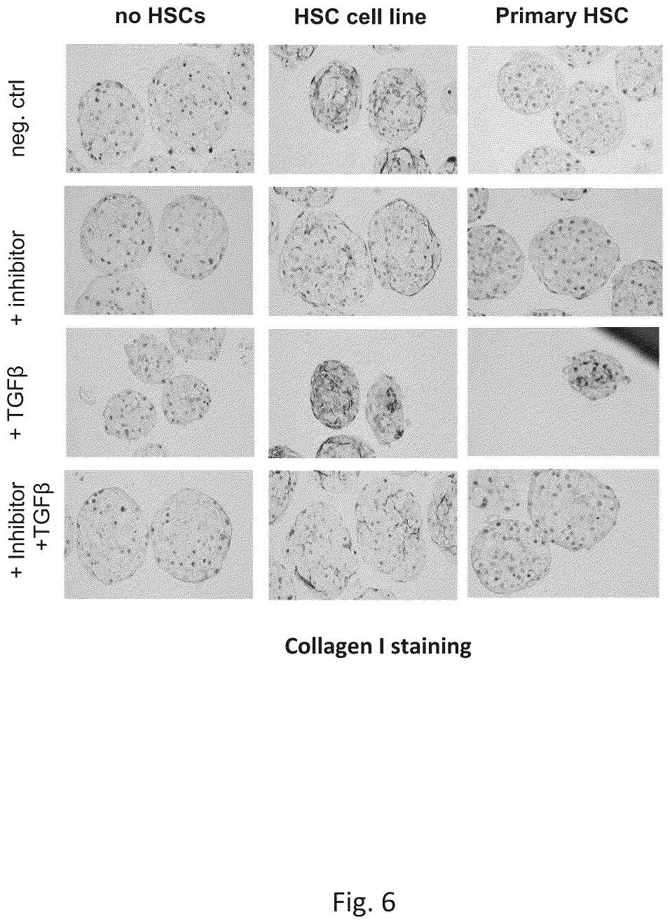

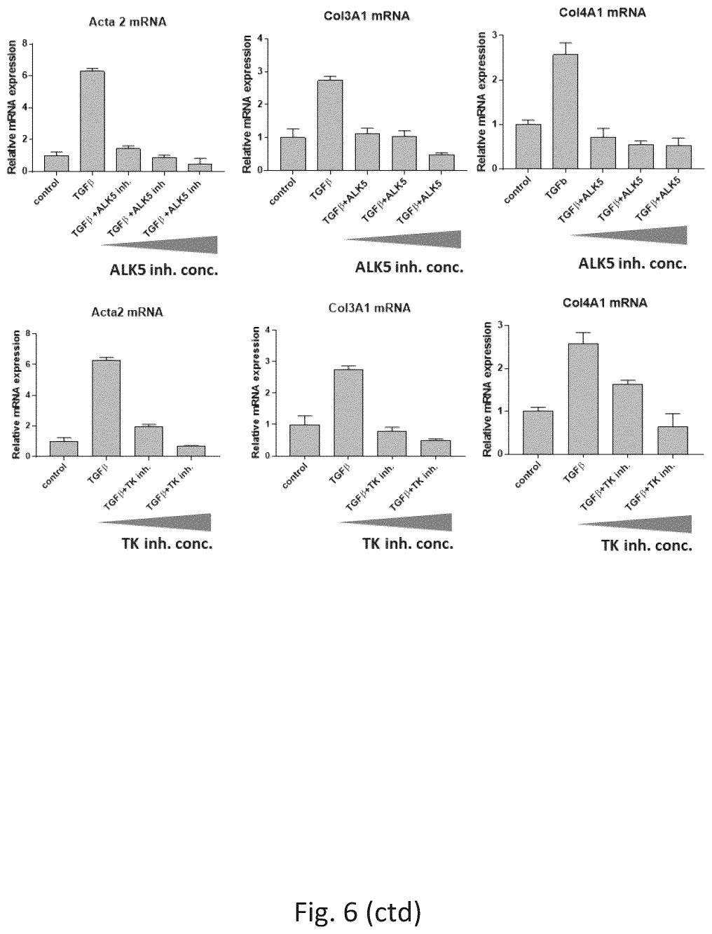

FIG. 6: Therapeutic effect of inhibition of pro-fibrotic effector pathways (TGF-βR, TK-R) was confirmed in triple co-culture liver MTs. Human liver MTs prepared form primary human hepatocytes, primary human Kupffer cells and primary human stellate cells where treated with TGF-β1 alone, or in combination with undisclosed inhibitors of i) TGF-β1 receptor, ii) ALK5, and iii) Tyrosine Kinase receptors. Progression of fibrosis by anti-fibrotic drug treatment in presence of pro-fibrotic agent TGF-β1 was clearly blunted as demonstrated by immunohistochemical staining of collagen I after 7 days of treatment. In another approach, the ALK5 inhibitor as well as the TK inhibitor showed a dose-dependent downregulation of the pro-fibrotic genes α-SMA (Acta 2), Collagen 3A1 and Collagen 4A1. In conclusion, these data demonstrate that the triple-culture model is suitable to assess anti-fibrotic properties of candidate compounds intended to be applied as prophylactic treatment.

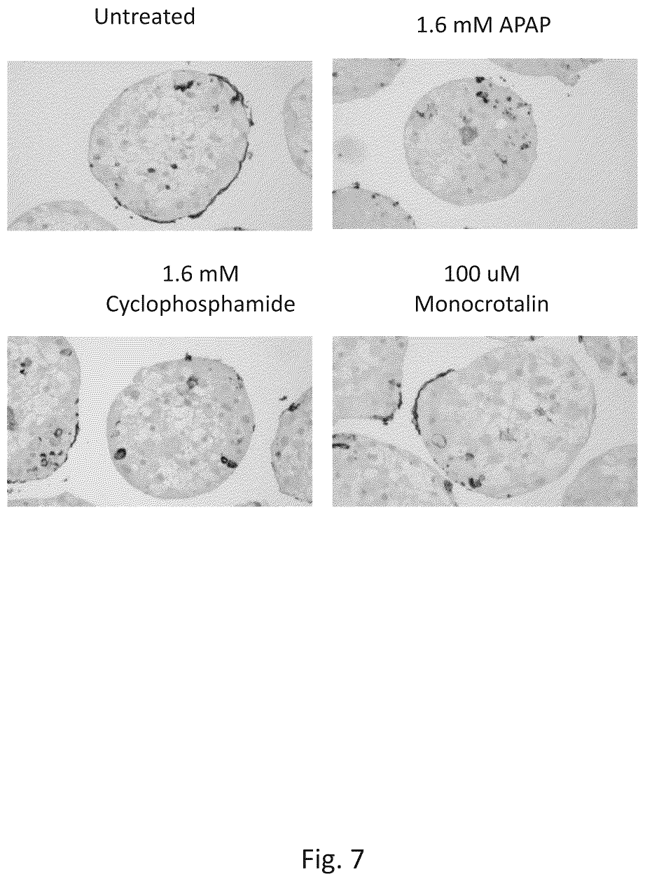

FIG. 7: Assessment of LEC-specific toxicity. LEC-coated microtissues were treated with Acetaminophen (APAP), Cyclosphosphamide and Monocrotaline for 5 days at the indicated concentrations, resulting in reduced LEC-presence on microtissues after treatment, as visualized by immunohistochemical staining for endothelial cell marker CD31.

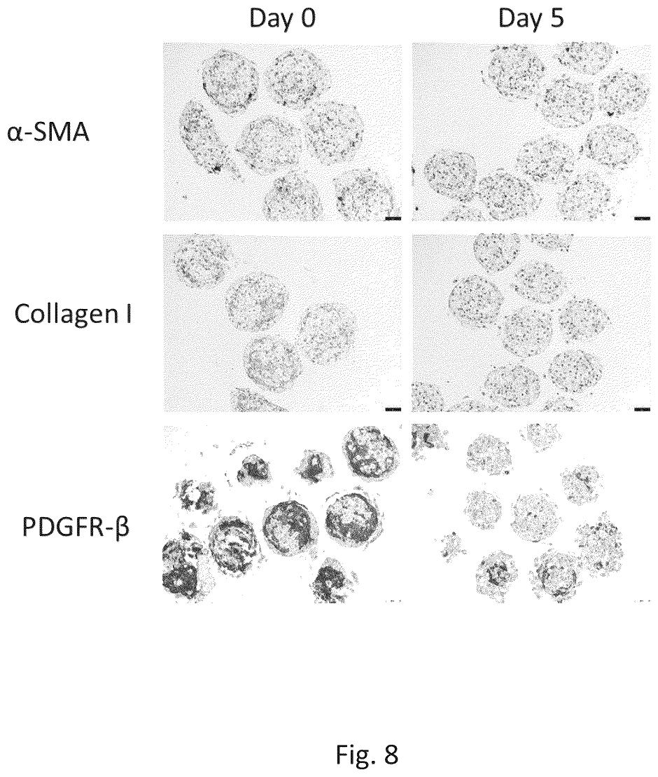

FIG. 8: Liver microtissues spontaneously revert to a more quiescent state after short term culture post production. Liver microtissues composed of primary hepatocytes, Kupffer cells and stellate cells where harvested 5 days after culture in specific maintenance medium and processed for immune histology. All three pro-fibrotic markers, α-SMA, Collagen I and PDGFR-β where less abundant than in MTs processed instantly after spheroid formation.

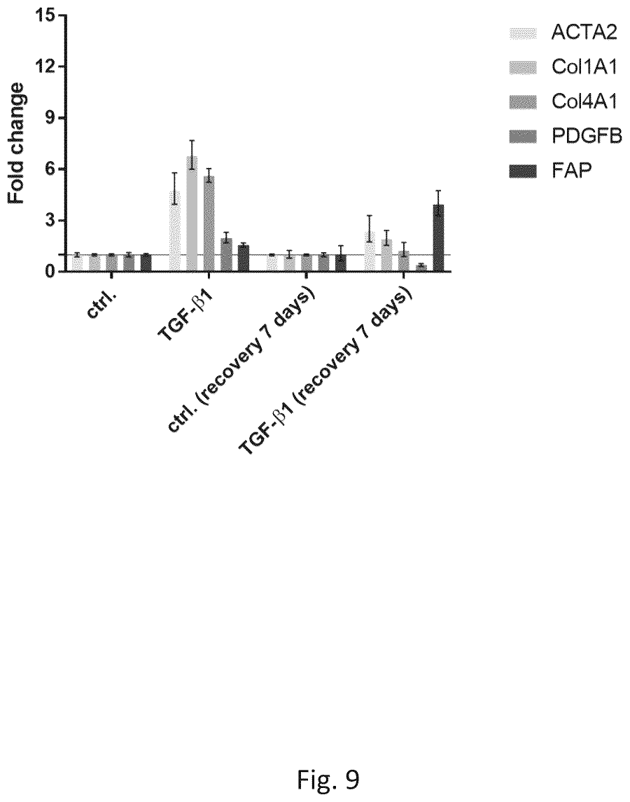

FIG. 9: Spontaneous reversion of fibrotic state. Liver microtissue composed of primary hepatocytes, Kupffer cells and stellate cells where treated with pro-fibrotic TGF-β1 for 7 days, upon which MTs were washed and further incubated in specified maintenance medium in absence of pro-fibrotic agents for another 7 days. Gene expression profiling for fibrotic markers revealed reduced transcript levels after at the end of the recovery phase when normalized to respective untreated controls.

### EXAMPLE 1: PRODUCTION OF TRIPLE CELL TYPE SPHEROIDAL MICROTISSUES (HEPATOCYTES, KUPFFER CELLS, STELLATE CELLS)

After growth in 2D culture homo- and heterotypic microtissues were produced in hanging drops using InSphero's GravityPLUS™ (InSphero) platform. For protocols see WO2015158777A1, the content of which is incorporated by reference herein
First, hepatocytes and hepatic stellate cells were detached from cell culture flasks
Therefore, medium was aspirated and the cell monolayer was washed with phosphate buffered saline (PBS)
Detaching of hepatocytes and HSCs was by Trypsin treatment
After optical confirmation of dissociation, thaw/wash medium was added, stopping the enzymatic reaction
Kupffer cells were thawed and directly used for MT production
Hepatocytes, HSCs and KCs were separately washed in thaw/wash medium by centrifugation
After the centrifugation step, the cells were resuspended in thaw/wash medium
Their cell number and viability was assessed
For MT production cell suspensions of the desired composition were prepared in human liver maintenance medium.

### EXAMPLE 2: COATING OF SPHEROIDAL MICROTISSUES WITH LIVER ENDOTHELIAL CELLS (LEC)

After The artificial spheroidal microtissue formation (Day 4), LEC have been thawed, washed with LEC Coating Medium—and centrifuged
In addition to the centrifugation step, the cells were resuspended in LEC Coating Medium and their cell number and viability has been assessed
For LEC seeding and coating of the spheroidal microtissues a cell suspension of the desired composition was prepared in LEC Coating Medium
spheroidal microtissues are being provided in suitable wells or vessel
5 μl of the LEC suspension are dispensed to each well or vessel.
LEC sink down and get in contact with the microtissues and the extracellular matrix surrounding them
After 2 days of incubation, microtissues are fully coated with LECs

### EXAMPLE 3: INDUCTION OF STEATOTIC PHENOTYPE BY LIPID LOADING OF LIVER MICROTISSUES

Dissolve FFA-free BSA at RT for 1h in H₂O for desired stock solution concentration
Dissolve desired amount of OA-Na in 1 mL 0.1 M NaOH-->at 70° C.
Dissolve desired amount of PA-Na 1 mL 0.1 M NaOH-->at 70°
Keep the FFAs on 70° C., while the BSA is placed on 37° C.
Mix FFA with BSA with a magnetic stirrer (for 30 min)
Dilute BSA with 0.1 M NaOH->vehicle control
Cool down to RT, sterile filter, aliquot and freeze.
For a final fatty acid (FA) solution dilute master stock in medium.
Replace medium of liver microtissues with FA-containing medium and incubate 2-7 days.

Oleate (OA), a monounsaturated omega-9 fatty acid, incorporates in hepatocytes within small lipid droplets (microvesicular steatosis), while palmitic acid (PA), saturated hexadecanoic acid, shows a rather macrovesicular phenotype (FIG. 3A1). Lipid accumulation is the first step for recapitulating NASH (Non-Alcoholic SteatoHepatitis) in vitro. Indeed, macrovesicular steatosis has been associated with lobular inflammation and fibrosis (Mulder et al., 2015).

Lipid loading shows a dose dependent effect (FIG. 3A2). While increasing concentrations of OA demonstrate saturation regarding lipid droplet density, PA administration showed reduced lipid incorporation at concentrations higher than PA3. This observation is suggestive of reduced metabolic activity or even compromised viability of liver microtissues at higher PA concentrations. A combination of OA/PA at a molar ratio of 2:1 shows a mixed phenotype with both features of micro- and macrovesicular steatosis. ATP measurement confirms the morphological observations, with decreased values in microtissues exposed to PA4 and higher, particularly after long-term treatment (FIG. 3A3). PA was less harmful when combined with OA, as demonstrated by ATP levels comparable to control samples (BSA). These data confirm the general concept of saturated fatty acids being capable of progressing NAFLD to NASH (Leamy et al., 2013), most likely by inducing ER-stress response, ROS formation, release of pro-inflammatory cytokines and activation of inflammasomes. Hereby Kupffer cells play an important role in promoting the inflammatory response (Dixon et al., 2012) up to the point of releasing chemokines for attracting circulating monocytes to further progress into an inflammatory state, paralleled by activation of stellate cells.

### EXAMPLE 4: INDUCTION OF INFLAMMATORY RESPONSE AND LIVER FIBROSIS

In order to assess the responsiveness towards prototypic inflammatory stimuli and pro-fibrotic stimuli on a gene-expression level, mono-cultured (HepaRG) and triple/co-cultured (HepaRG THP1/hTERT-HSC) liver MT were stimulated with inflammatory inducers (LPS or TNF-α) and the pro-fibrotic cytokine TGF-β1 to assess the cellular responses. Significant differences in the responses of the two types of MT were observed (FIG. 4). The treatment with LPS and TNF-α resulted in increased cellular viability of co-cultured microtissues, whereas TGF-β1 decreased viability after 14 days of treatment. Analysis of gene expression changes after treatments showed a strong induction of the pro-fibrotic markers Collagen I and IV, fibronectin, CD44 and TGF-β1 in co-cultured MTs, indicating presence of extracellular matrix protein transcription and further stimulation of pro-fibrotic conditions. HepaRG monocultures, however, exhibited only moderate or no response towards pro-fibrotic stimuli. For example, mRNA of collagen I was only detected in the co-culture system, but not in the monocultures. Interestingly, monocultures were strongly responsive towards LPS and TNF-α for IL-6 gene induction, demonstrating the responsiveness for inflammatory stimuli of these hepatocyte-like cells in spheroidal microtissues. TGF-β1 did not induce IL-6 gene expression in monocultures, but was observed in co-cultures, suggesting monocytes as the predominant source of IL-6 (FIG. 4).

### EXAMPLE 5: A METHOD TO WHICH EMPLOYS THE TRIPLE/QUADRUPLE LIVER MICROTISSUE MODEL TO ASSESS ADVERSE LIVER EFFECTS SUCH AS FIBROSIS AND CIRRHOSIS

In order to test whether the described in vitro model is capable of recapitulating induction of liver fibrosis upon treatment with clinically relevant compounds, we tested the pro-fibrotic chemicals methotrexate (MTX) and Thiocetamide (TAA), which were both shown to cause hepatic fibrosis in vivo. Exposure of co-cultured Microtissues to MTX and TAA over 14 days showed a dose-dependent decrease of MT viability with both MTX and TAA (FIG. 5). Gene expression analysis showed reproducible, also dose-dependent transcriptional induction of collagen I, collagen IV, fibronectin I and CD44 (FIG. 5).

### EXAMPLE 6: A METHOD TO ASSESS ADVERSE EFFECTS ON LIVER ENDOTHELIAL CELLS

Spheroidal liver microtissues containing hepatocytes and Kupffer cells were coated with liver endothelial cells (LEC) and subjected to 5 days treatment with sub-toxic doses of Acetaminophen (APAP), Cyclophosphamide and Monocrotaline (FIG. 7). After the treatment, microtissues were collected, formalin fixed and paraffin embedded, sliced and stained by immunohistochemistry for the LEC-marker CD31. The marker was present in the untreated control, confirming the coating of the LEC of the spheroidal Liver Microtissue. After treatment with the LEC-toxicants acetaminophen, cyclosphosphamide and monocrotalin, however, the CD31-marker protein was heavily reduced or completely absent. This suggests that these compounds indeed are toxic to LECs at the tested concentration.

### EXAMPLE 7: INHIBITION OF FIBROSIS PROGRESSION (FIG. 6)

Human liver microtissues (+Kupffer cells) without stellate cells and with either a stellate cell line or primary human stellate cells were treated with a fibrosis inducer (TGF-β1) and in combination with an undisclosed fibrosis inhibitor. Histological analysis after 7 days treatment demonstrates significant reduction of basal fibrosis represented by diminished staining for collagen I in cell line derived HSC cultures compared to the untreated control. Even more pronounced is the difference between TGF-β1 treated stellate cell comprising microtissues and TGF-β1/inhibitor treated cultures. While TGF-β1 treated microtissues display a severe fibrotic phenotype with collagen deposition, co-treatment nearly completely inhibited the fibrotic phenotype. When co-treated with TGF-β1, the anti-fibrotic properties of another two compounds acting on different targets involved in the progression of fibrosis, i.e. an ALK5 inhibitor or a Tyrosine Kinase (TK) inhibitor, could also be demonstrated on the basis of the blunted induction of transcripts of the fibrotic markers α-SMA, Collagen 3A1 and Collagen 4A1. This again demonstrates the prevention of the fibrotic phenotype by pharmacological intervention and thus the applicability of 3-cell type liver microtissues as a model for testing anti-fibrotic properties of putative therapeutic compounds.

### EXAMPLE 8: SPONTANEOUS REVERSION OF STELLATE CELL ACTIVATION OR FIBROTIC PHENOTYPE

Stellate cells do show explicit dynamic behavior acting in response to tissue injury (e.g. trauma, viral infection, inflammatory processes, or ischemia) by transdifferentiating from a lipocyte-like cell into a contractile and highly proliferative myofibroblast-like cell which produce a variety of extracellular matrix components as part of the scar-formation process. This very well reflects one of their primary biological functions, namely the initiation of tissue repair upon various types of tissue insult. Therefore stellate cells are very sensitive of environmental alterations beyond normal physiological limits. This also is manifested under in vitro conditions usually applied during the cell expansion phase, namely culturing stellate cells on conventional cell culture plastic dishes. This by far does not represent their natural environment which consequently leads to a change in their phenotype towards an activated state reflecting the repair mode similar but not identical (see De Mincis et al., 2007) to in vivo conditions after tissue injury. Stellate cells revert to their normal quiescent state once tissue injury has been resolved (Kisseleva et al., 2012). This exact dynamic behavior is also observed in the course of in vitro expansion and subsequent 3D co-culture in combination with hepatocytes and Kupffer cells, where stellates undergo temporary activation manifested by elevated pro-fibrotic markers (e.g. α-SMA, Collagen I, PDGFR-β). Reversion can be achieved within a few days after set-up of 3D culture simply by co-culture with hepatocytes and Kupffer cells in 3D-configuration in the absence of any stressor signal, i.e. in regular culture conditions (37° C., 5% CO₂, in maintenance medium optimized for liver 3D culture), as illustrated in FIG. 8. This is the first time such spontaneous reversion from an activated state of stellate cells to a more quiescent state phenotype is observed in vitro not requiring particular treatment with medium additives (growth factors, such as FGF2, EGF, retinol and free fatty acids) as recently described by Taghdouini et al., (2015). As this spontaneous type of reversion was not observed in in 2D culture set-up or 3D mono-culture involving only stellate cells, it strongly suggests that only the interplay of stellate cells with other livers cells, mainly hepatocytes and Kupffer cells, fostered by the 3D spheroid configuration allows this dynamic, bi-phasic behavior of this cell type.

The strength of the proposed liver in vitro culture format for the study of the dynamic behavior upon pharmacological, inflammatory or dietary stressors in the progression of fatty liver disease has also been demonstrated in the case of treatment with the pro-fibrotic cytokine TGF-β1. Gene expression levels of fibrotic markers (ACTA2, Col1A1, Col4A1, PDGFB) were clearly unregulated after stimulation with TGF-β1, but reversed to almost baseline levels within 7 days after TGF-β1 treatment was abolished (FIG. 9). This observation is indicative of the capacity of the model system to revert back from a pathophysiological to a normal physiological state by resolving the fibrotic phenotype, a fundamental prerequisite for in vitro test-system to be applied for testing therapeutic strategies after the onset of the disease. Interestingly aside from declined gene expression levels of the fibrotic markers after the wash-out phase, the simultaneous upregulation of the fibroblast activation protein (FAP) confirms the reversion from the diseased into a more healthy state. FAP has recently been described to be involved in the clearance in collagen catabolism and clearance is believed to be important for resolving scar after tissue injury and remodeling (Fan et. al., 2016).

### EXAMPLE 9: METABOLIC LIVER DISEASE TESTING PLATFORM (FIG. 1A, 1B)

Based on the described experiments and related observations an in vitro platform is proposed, which is capable to allow following investigative modes:
1. Study of disease progression and testing of anti-NASH/Fibrosis therapeutic or preventive strategies by pharmacological and/or dietary interventions, starting from different entry points:
   Liver model with pro-activated stellate cells (T₁, right after tissue formation, process [a], described in example 1)
   Liver model at basal state, after reversion from activated post-production state (process [b], described in example 8), or further progressed to either of the following states:
      Liver model with insulin resistant state (process [b′]) introduced by
         High glucose/fructose
         High insulin
         High free fatty acids (SFA>MUFA>PUFA)
         Adipocyte derived cytokines TNF-α, IL-1β, and IL-6
         Resistin
         Glucocorticoids
         or a combination of above
      Liver model with steatotic state, (process [c], described in example 3).
         MUFA:SFA molar ratio ≥1
      Liver model with inflammatory state, (process [d], described in example 4).
      Liver model with fibrotic state, (process [e], described in example 4 and 5).
      Liver model with NASH/Fibrosis phenotype (option 1), (by sequential or simultaneous application of process [b′], [c], [d], and [e]).
      Liver model with NASH/Fibrosis phenotype (option 2), by treatment with pro-inflammatory dietary free fatty acid composition (process [f])
         SFA only
         or MUFA:SFA molar ratio <1
      Most common SFAs in human diet are: Stearic acid, Palmitic acid, Myristic acid, Lauric acid, Capric acid, Caprylic acid, Caproic acid Most common MUFAs in human diet are: Oleic acid, Palmitoleic acid
   Liver model at basal state or any of the above disease models in co-culture with either type of inflammatory cells
      NK, NKT or iNKT cells,
      T lymphocytes,
      polymorphonuclear cells
      CD8 lymphocytes
      Peripheral Blood Mononuclear Cells (PBMCs)
      any of above inflammatory cells possibly haplotype-matched to liver cells used for tissue formation
      as static co-culture or in a microfluidic device allowing free floating of inflammatory cells with immobilized liver microtissues
2. Liver model at basal state or any of the above models representing advanced states of diseased liver for safety assessment of drug candidates with suspected liver toxic liabilities, as described in example 5 and 6.

The versatility of the platform allows different modes of implementation or commercialization:
2. Provision of aforementioned basal liver models and disease models as assay ready system, including
   a. Basal maintenance medium
   b. Diseased state-specific media
   c. QC certification
   d. Reference test compounds
3. Provision of aforementioned basal liver models and supplements for the induction of any of above mentioned disease states as assay-ready system, including
   a. Basal maintenance medium
   b. QC certification
   c. Reference test compounds

### REFERENCES

van Grunsven L. A., Advanced Drug Delivery Reviews. 2017; 121 pp: 133-146
Kelm et al., J Biotechnol. 2005; 118 pp: 213-29
Leite S. et al., Biomaterials. 2016; 78 pp: 1-10
Liedtke C et al., Fibrogenesis Tissue Repair. 2013; 6: 19
Ming-Hui Fan et. al., J Biol Chem. 2016; 291(15): 8070-8089
Puche, Saiman, & Friedman, Compr Physiol. 2013; 3(4):1473-92
Taghdouini et al., Fibrogenesis Tissue Repair. 2015; 8:14
Nguyen D G et al., PLoS ONE 11(7) (2016): e0158674
The Importance of Multicellularity in Liver Homeostasis and Injury, 2015, Organovo
Duarte et al., BioMed Research International Volume 2015 (2015), Article ID 984578
Mulder et al., Inflamm Cell Signal 2015; 2: e804
Leamy et al., Prog Lipid Res. 2013 January; 52(1): 165-174
Dixon et al., Lab Invest. 2012 May; 92(5): 713-723
Fan et. al., J Biol Chem. 2016; 291(15): 8070-8089
Taghdouini et al., Fibrogenesis Tissue Repair. 2015; 8:14
Kisseleva et al., Proc Natl Acad Sci. 2012; 109(24): 9448-53
DeMincis et al., Gastroenterology. 2007 May; 132(5):1937-46

1. A method of preparing an artificial spheroidal microtissue, the artificial spheroidal microtissue comprising primary human hepatocytes, primary human hepatic stellate cells and primary human Kupffer cells, the method comprising:
co-culturing primary human hepatocytes, Kupffer and stellate cells in maintenance medium for liver 3D culture without any of a growth factor, retinol or a free fatty acid supplementation from culture onset, and obtaining the artificial spheroidal microtissue 5 days after culture onset or later, such that the artificial spheroidal microtissue adopts a non-fibrotic normal physiological state.

2. The method according to claim 1, wherein the microtissue
a) has a diameter of between ≥30 μm and ≤500 μm, or
b) comprises between ≥500 and ≤10000 cells in total.

3. The method according to claim 1, wherein the microtissue further comprises sinusoidal endothelial cells.

4. The method according to claim 3, wherein the sinusoidal endothelial cells form a cell layer around said microtissue comprising primary human hepatocytes, primary human hepatic stellate cells, and primary human Kupffer cells.

5. The method according to claim 1, further comprising harvesting the obtained artificial spheroidal microtissue.

6. The method according to claim 1, wherein the microtissue represents a liver model at basal state.

7. The method according to claim 6, wherein the microtissue is progressed to at least one of the states selected from the list consisting of:
a) insulin resistant state,
b) steatotic state,
c) inflammatory state,
d) fibrotic state,
e) NASH/Fibrosis phenotype, option 1,
f) NASH/Fibrosis phenotype, option 2.

8. The method according to claim 1, further comprising
(a) producing or providing a microtissue comprising human hepatocytes, human hepatic stellate cells and human Kupffer cells,
(b) dispensing said suspension into wells or vessels comprising one or more microtissues of step (a) and
(c) incubating said wells or vessels.

9. The method according to claim 1, further comprising
(d) producing or providing a microtissue comprising human hepatocytes, human hepatic stellate cells and human Kupffer cells,
(e) producing or providing a suspension comprising sinusoidal endothelial cells,
(f) dispensing said suspension into wells or vessels comprising one or more microtissues of step (a) and
(g) incubating said wells or vessels to let sinusoidal endothelial cells adhere to the microtissues.

10. The method according to claim 1, wherein co-culturing primary human hepatocytes, Kupffer and stellate cells is performed in a hanging drop system or a low adherence well.

11. The method according to claim 1, comprising obtaining the artificial spheroidal microtissue 7 days after culture onset or later.

12. The method according to claim 1, wherein the obtained microtissue shows reduced abundancy of at least one profibrotic marker selected from the group consisting of a-SMA, Collagen I and PDGFR-β compared to a microtissue harvested instantly after spheroid formation.

13. The method according to claim 1, further comprising at least one subsequent step selected from the group consisting of: drug efficacy and/or toxicity screenings, investigative/mechanistic toxicology, target discovery/identification, drug repositioning studies, and/or pharmacokinetics and pharmacodynamics assays.

14. An assay method for testing compounds
suspected for promoting, causing, or increasing the risk of NAFLD/NASH/Fibrosis, or
for their potency to reduce the risk, alleviate the symptoms, treat the causes of NAFLD/NASH/Fibrosis or to reverse the pathological states of NAFLD/NASH/Fibrosis,
in an artificial spheroidal microtissue comprising primary human hepatocytes, primary human hepatic stellate cells and primary human Kupffer cells, the artificial spheroidal microtissue being in a non-fibrotic normal physiological state,
wherein the method comprises the steps of:
a) treating with one or more test compounds suspected of promoting, causing, or increasing the risk of, NAFLD/NASH/Fibrosis;
b) in parallel or subsequently treating with one or more test compounds intended to reverse NAFLD/NASH/Fibrosis; and
c) carrying out an endpoint analysis of at least one genetic, physiologic and/or morphologic parameter relevant to the respective liver pathology.

15. The method according to claim 14, wherein the endpoint analysis refers to at least one parameter selected from the group consisting of: release of one or more biomarkers, lipid content of the microtissue cells, histology of the microtissue and its cells (disrupted/non disrupted), RNA expression marker such as alpha-smooth muscle actin, COL1A1, vimentin or TIMP-1, size or volume of the microtissue, and/or mitochondrial activity.
